SEQUENCE LISTING (12 sequences)

SEQ ID NO: 1 - protein, 976 aa, Homo sapiens [sequence omitted]

SEQ ID NO: 2 - protein, 977 aa, Homo sapiens
MRGARGAWDFLCVLLLLLRVQTGSSQPSVSPGEPSPPSIHPGKSDLIVRVGDEIRLLCTDPGFVKWTFEILDETNENKQNEWITEKAEATNTGKYTCTNKHGLSNSIYVFVRDPAKLFLVDRSLYGKEDNDTLVRCPLTDPEVTNYSLKGCQGKPLPKDLRFIPDPKAGIMIKSVKRAYHRLCLHCSVDQEGKSVLSEKFILKVRPAFKAVPVVSVSKASYLLREGEEFTVTCTIKDVSSSVYSTWKRENSQQTKLQEKYNSWHHGDFNYERQATLTISSARVNDSGVFMCYANNTFGSANVTTTLEVVDKGFINIFPMINTTVFVNDGENVDLIVEYEAFPKPEHQQWIYMNRTFTDKWEDYPKSENESNIRYVSELHLTRLKGTEGGTYTFLVSNSDVNAAIAFNVYVNTKPEILTYDRLVNGMLQCVAAGFPEPTIDWYFCPGTEQRCSASVLPVDVQTLNSSGPPFGKLVVQSSIDSSAFKHNGTVECKAYNDVGKTSAYFNFAFKGNNKEQIHPHTLFTPLLIGFVIVAGMMCIIVMILTYKYLQKPMYEVQWKVVEEINGNNYVYIDPTQLPYDHKWEFPRNRLSFGKTLGAGAFGKVVEATAYGLIKSDAAMTVAVKMLKPSAHLTEREALMSELKVLSYLGNHMNIVNLLGACTIGGPTLVITEYCCYGDLLNFLRRKRDSFICSKQEDHAEAALYKNLLHSKESSCSDSTNEYMDMKPGVSYVVPTKADKRRSVRIGSYIERDVTPAIMEDDELALDLEDLLSFSYQVAKGMAFLASKNCIHRDLAARNILLTHGRITKICDFGLARDIKNDSNYVVKGNARLPVKWMAPESIFNCVYTFESDVWSYGIFLWELFSLGSSPYPGMPVDSKFYKMIKEGFRMLSPEHAPAEMYDIMKTCWDADPLKRPTFKQIVQLIEKQISESTNHIYSNLANCSPNRQKPVVDHSVRINSVGSTASSSQPLLVHDDV

SEQ ID NO: 3 - protein, 975 aa, Homo sapiens
MRGARGAWDFLCVLLLLLRVQTGSSQPSVSPGEPSPPSIHPGKSDLIVRVGDEIRLLCTDPGFVKWTFEILDETNENKQNEWITEKAEATNTGKYTCTNKHGLSNSIYVFVRDPAKLFLVDRSLYGKEDNDTLVRCPLTDPEVTNYSLKGCQGKPLPKDLRFIPDPKAGIMIKSVKRAYHRLCLHCSVDQEGKSVLSEKFILKVRPAFKAVPVVSVSKASYLLREGEEFTVTCTIKDVSSSVYSTWKRENSQTKLQEKYNSWHHGDFNYERQATLTISSARVNDSGVFMCYANNTFGSANVTTTLEVVDKGFINIFPMINTTVFVNDGENVDLIVEYEAFPKPEHQQWIYMNRTFTDKWEDYPKSENESNIRYVSELHLTRLKGTEGGTYTFLVSNSDVNAAIAFNVYVNTKPEILTYDRLVNGMLQCVAAGFPEPTIDWYFCPGTEQRCSASVLPVDVQTLNSSGPPFGKLVVQSSIDSSAFKHNGTVECKAYNDVGKTSAYFNFAFKGNNKEQIHPHTLFTPLLIGFVIVAGMMCIIVMILTYKYLQKPMYEVQWKVVEEINGNNYVYIDPTQLPYDHKWEFPRNRLSFGKTLGAGAFGKVVEATAYGLIKSDAAMTVAVKMLKPSAHLTEREALMSELKVLSYLGNHMNIVNLLGACTIGGPTLVITEYCCYGDLLNFLRRKRDSFICSKQEDHAEAALYKNLLHSKESSCDSTNEYMDMKPGVSYVVPTKADKRRSVRIGSYIERDVTPAIMEDDELALDLEDLLSFSYQVAKGMAFLASKNCIHRDLAARNILLTHGRITKICDFGLARDIKNDSNYVVKGNARLPVKWMAPESIFNCVYTFESDVWSYGIFLWELFSLGSSPYPGMPVDSKFYKMIKEGFRMLSPEHAPAEMYDIMKTCWDADPLKRPTFKQIVQLIEKQISESTNHIYSNLANCSPNRQKPVVDHSVRINSVGSTASSSQPLLVHDDV

SEQ ID NO: 4 - protein, 976 aa, Homo sapiens
MRGARGAWDFLCVLLLLLRVQTGSSQPSVSPGEPSPPSIHPGKSDLIVRVGDEIRLLCTDPGFVKWTFEILDETNENKQNEWITEKAEATNTGKYTCTNKHGLSNSIYVFVRDPAKLFLVDRSLYGKEDNDTLVRCPLTDPEVTNYSLKGCQGKPLPKDLRFIPDPKAGIMIKSVKRAYHRLCLHCSVDQEGKSVLSEKFILKVRPAFKAVPVVSVSKASYLLREGEEFTVTCTIKDVSSSVYSTWKRENSQQTKLQEKYNSWHHGDFNYERQATLTISSARVNDSGVFMCYANNTFGSANVTTTLEVVDKGFINIFPMINTTVFVNDGENVDLIVEYEAFPKPEHQQWIYMNRTFTDKWEDYPKSENESNIRYVSELHLTRLKGTEGGTYTFLVSNSDVNAAIAFNVYVNTKPEILTYDRLVNGMLQCVAAGFPEPTIDWYFCPGTEQRCSASVLPVDVQTLNSSGPPFGKLVVQSSIDSSAFKHNGTVECKAYNDVGKTSAYFNFAFKGNNKEQIHPHTLFTPLLIGFVIVAGMMCIIVMILTYKYLQKPMYEVQWKVVEEINGNNYVYIDPTQLPYDHKWEFPRNRLSFGKTLGAGAFGKVVEATAYGLIKSDAAMTVAVKMLKPSAHLTEREALMSELKVLSYLGNHMNIVNLLGACTIGGPTLVITEYCCYGDLLNFLRRKRDSFICSKQEDHAEAALYKNLLHSKESSCDSTNEYMDMKPGVSYVVPTKADKRRSVRIGSYIERDVTPAIMEDDELALDLEDLLSFSYQVAKGMAFLASKNCIHRDLAARNILLTHGRITKICDFGLARDIKNDSNYVVKGNARLPVKWMAPESIFNCVYTFESDVWSYGIFLWELFSLGSSPYPGMPVDSKFYKMIKEGFRMLSPEHAPAEMYDIMKTCWDADPLKRPTFKQIVQLIEKQISESTNHIYSNLANCSPNRQKPVVDHSVRINSVGSTASSSQPLLVHDDV

SEQ ID NO: 5 - protein, 25 aa, Mus musculus
MRGARGAWDLLCVLLVLLRGQTATS

SEQ ID NO: 6 - protein, 452 aa, Mus musculus
LLIGFVVAAGAMGIIVMVLTYKYLQKPMYEVQWKVVEEINGNNYVYIDPTQLPYDHKWEFPRNRLSFGKTLGAGAFGKVVEATAYGLIKSDAAMTVAVKMLKPSAHLTEREALMSELKVLSYLGNHMNIVNLLGACTVGGPTLVITEYCCYGDLLNFLRRKRDSFIFSKQEEQAEAALYKNLLHSTEPSCDSSNEYMDMKPGVSYVVPTKTDKRRSARIDSYIERDVTPAIMEDDELALDLDDLLSFSYQVAKGMAFLASKNCIHRDLAARNILLTHGRITKICDFGLARDIRNDSNYVVKGNARLPVKWMAPESIFSCVYTFESDVWSYGIFLWELFSLGSSPYPGMPVDSKFYKMIKEGFRMVSPEHAPAEMYDVMKTCWDADPLKRPTFKQVVQLIEKQISDSTKHIYSNLANCNPNPENPVVVDHSVRVNSVGSSASSTQPLLVHEDA

SEQ ID NO: 7 - protein, 499 aa, Homo sapiens
QPSVSPGEPSPPSIHPGKSDLIVRVGDEIRLLCTDPGFVKWTFEILDETNENKQNEWITEKAEATNTGKYTCTNKHGLSNSIYVFVRDPAKLFLVDRSLYGKEDNDTLVRCPLTDPEVTNYSLKGCQGKPLPKDLRFIPDPKAGIMIKSVKRAYHRLCLHCSVDQEGKSVLSEKFILKVRPAFKAVPVVSVSKASYLLREGEEFTVTCTIKDVSSSVYSTWKRENSQTKLQEKYNSWHHGDFNYERQATLTISSARVNDSGVFMCYANNTFGSANVTTTLEVVDKGFINIFPMINTTVFVNDGENVDLIVEYEAFPKPEHQQWIYMNRTFTDKWEDYPKSENESNIRYVSELHLTRLKGTEGGTYTFLVSNSDVNAAIAFNVYVNTKPEILTYDRLVNGMLQCVAAGFPEPTIDWYFCPGTEQRCSASVLPVDVQTLNSSGPPFGKLVVQSSIDSSAFKHNGTVECKAYNDVGKTSAYFNFAFKGNNKEQIHPHTLFTP

SEQ ID NO: 8 - protein, 500 aa, Homo sapiens
QPSVSPGEPSPPSIHPGKSDLIVRVGDEIRLLCTDPGFVKWTFEILDETNENKQNEWITEKAEATNTGKYTCTNKHGLSNSIYVFVRDPAKLFLVDRSLYGKEDNDTLVRCPLTDPEVTNYSLKGCQGKPLPKDLRFIPDPKAGIMIKSVKRAYHRLCLHCSVDQEGKSVLSEKFILKVRPAFKAVPVVSVSKASYLLREGEEFTVTCTIKDVSSSVYSTWKRENSQQTKLQEKYNSWHHGDFNYERQATLTISSARVNDSGVFMCYANNTFGSANVTTTLEVVDKGFINIFPMINTTVFVNDGENVDLIVEYEAFPKPEHQQWIYMNRTFTDKWEDYPKSENESNIRYVSELHLTRLKGTEGGTYTFLVSNSDVNAAIAFNVYVNTKPEILTYDRLVNGMLQCVAAGFPEPTIDWYFCPGTEQRCSASVLPVDVQTLNSSGPPFGKLVVQSSIDSSAFKHNGTVECKAYNDVGKTSAYFNFAFKGNNKEQIHPHTLFTP

SEQ ID NO: 9 - protein, 499 aa, Homo sapiens; identical to SEQ ID NO: 7

SEQ ID NO: 10 - protein, 500 aa, Homo sapiens; identical to SEQ ID NO: 8

SEQ ID NO: 11 - protein, 976 aa, synthetic construct
MRGARGAWDLLCVLLVLLRGQTATSQPSVSPGEPSPPSIHPGKSDLIVRVGDEIRLLCTDPGFVKWTFEILDETNENKQNEWITEKAEATNTGKYTCTNKHGLSNSIYVFVRDPAKLFLVDRSLYGKEDNDTLVRCPLTDPEVTNYSLKGCQGKPLPKDLRFIPDPKAGIMIKSVKRAYHRLCLHCSVDQEGKSVLSEKFILKVRPAFKAVPVVSVSKASYLLREGEEFTVTCTIKDVSSSVYSTWKRENSQTKLQEKYNSWHHGDFNYERQATLTISSARVNDSGVFMCYANNTFGSANVTTTLEVVDKGFINIFPMINTTVFVNDGENVDLIVEYEAFPKPEHQQWIYMNRTFTDKWEDYPKSENESNIRYVSELHLTRLKGTEGGTYTFLVSNSDVNAAIAFNVYVNTKPEILTYDRLVNGMLQCVAAGFPEPTIDWYFCPGTEQRCSASVLPVDVQTLNSSGPPFGKLVVQSSIDSSAFKHNGTVECKAYNDVGKTSAYFNFAFKGNNKEQIHPHTLFTPLLIGFVVAAGAMGIIVMVLTYKYLQKPMYEVQWKVVEEINGNNYVYIDPTQLPYDHKWEFPRNRLSFGKTLGAGAFGKVVEATAYGLIKSDAAMTVAVKMLKPSAHLTEREALMSELKVLSYLGNHMNIVNLLGACTVGGPTLVITEYCCYGDLLNFLRRKRDSFIFSKQEEQAEAALYKNLLHSTEPSCDSSNEYMDMKPGVSYVVPTKTDKRRSARIDSYIERDVTPAIMEDDELALDLDDLLSFSYQVAKGMAFLASKNCIHRDLAARNILLTHGRITKICDFGLARDIRNDSNYVVKGNARLPVKWMAPESIFSCVYTFESDVWSYGIFLWELFSLGSSPYPGMPVDSKFYKMIKEGFRMVSPEHAPAEMYDVMKTCWDADPLKRPTFKQVVQLIEKQISDSTKHIYSNLANCNPNPENPVVVDHSVRVNSVGSSASSTQPLLVHEDA

SEQ ID NO: 12 - DNA, 288 bp, Mus musculus
  source 1..288: mol_type = genomic DNA
ccacgtctcagccatctgcaagtccaggggagccgtctccgccatccatccatccagcacaatcagagttaatagttgaagctggcgacaccctcagcctgacgtgcattgatcccgactttgtcagatggactttcaagacctatttcaatgaaatggttgagaataaaaaaaatgaatggatccaggaaaaagccgaggccactcgcacgggcacatacacgtgcagcaacagcaatggcctcacgagttctatttacgtgtttgttagagatcctgccaaacttt

(19) United States
(12) Patent Application Publication
(10) Pub. No.: US 2026/0053122 A1
(43) Pub. Date: Feb. 26, 2026

(54) TRANSGENIC ANIMALS EXPRESSING CHIMERIC C-KIT PROTEIN

(71) Applicant: Jasper Therapeutics, Inc., Redwood City, CA (US)

(72) Inventor: Wendy PANG, Palo Alto, CA (US)

(21) Appl. No.: 19/103,734

(22) PCT Filed: Aug. 15, 2023
(86) PCT No.: PCT/US2023/072253

§ 371 Date: Feb. 13, 2025

Related U.S. Application Data
(60) Provisional application No. 63/371,489, filed on Aug. 15, 2022.

(51) Int. Cl. A01K 67/0278; A61K 49/00; C07K 16/28; C12N 9/12

(52) U.S. Cl. CPC A01K 67/0278; A61K 49/0008; C07K 16/2803; C12N 9/12; C12Y 207/10001; A01K 2217/052; A01K 2227/105; A01K 2267/03

(57) ABSTRACT

The present disclosure describes methods of generating a transgenic cell or mouse that expresses a chimeric c-KIT protein capable of expression on mouse cells and binding to mouse stem cell factor (SCF) which can be inhibited by anti-human c-KIT inhibitors. The transgenic cells and mice can be used to test candidate anti-human c-KIT inhibitors.

### CROSS REFERENCE TO RELATED APPLICATIONS

This application claims the benefit under 35 U.S.C. § 119(e) of the U.S. Provisional Application Ser. No. 63/371,489, filed Aug. 15, 2022, the content of which is hereby incorporated by reference in its entirety.

### REFERENCE TO AN ELECTRONIC SEQUENCE LISTING

The contents of the electronic sequence listing (350546.xml; Size: 18,701 bytes; and Date of Creation: Aug. 15, 2023) is herein incorporated by reference in its entirety.

### BACKGROUND

The tyrosine-protein kinase KIT is also known as c-KIT, CD117 (cluster of differentiation 117) or SCFR (mast/stem cell growth factor receptor). C-KIT is expressed on the surface of hematopoietic stem cells, mast cells, neural crest-derived melanocytes, and germ cells. C-KIT binds to stem cell factor (SCF) and forms a dimer that activates its intrinsic tyrosine kinase activity, which in turn phosphorylates and activates signal transduction molecules that propagate the signal in the cell.

Anti-c-KIT antibodies are being developed, which can be useful for inhibiting c-KIT-mediated signaling, leading to ablation of hematopoietic stem cells in subject. Such ablation is helpful to ensure efficacy of subsequent hematopoietic stem cell engraftments. Development of such antibodies can be facilitated with animal models that express the human c-KIT protein. Introduction of a cDNA encoding the human c-KIT protein to an animal, however, may not be successful. For instance, the human c-KIT protein may not be expressed in the desired cells, localized correctly, or undergo the correct posttranslational modifications. Moreover, the human c-KIT protein, if activated, may not trigger the correct downstream signaling in the animal cells.

The human c-KIT protein has an extracellular domain (ECD) with 5 Ig-like domains (D1-D5), which are implicated in ligand binding and homodimerization, a transmembrane domain, and an intracellular domain (ICD). Protein processing of c-KIT includes N-linked glycosylation and phosphorylation on both tyrosine and serine residues. When SCF dimers bind to c-KIT, the binding induce c-KIT dimerization, conformational change of both ECD and ICD, ICD trans-phosphorylation and binding of various partners.

Design of human-animal chimeric c-KIT proteins also presents significant challenges. For instance, human and mouse c-KIT proteins have 90% sequence similarity. However, protein glycosylation and the conformational changes of both ECD and ICD required for activation could be impaired in a chimeric protein. Moreover, it was unknown how a chimeric protein would function to induce cell proliferation in cells with different levels of endogenous c-KIT expression, and how they would respond to mouse SCF.

### SUMMARY

The present disclosure describes methods of generating a transgenic cell or mouse that expresses a chimeric c-KIT protein capable of expression on mouse cells and binding to mouse stem cell factor (SCF). Such binding can be inhibited by anti-human c-KIT inhibitors, and therefore serves as a platform for inhibitor testing.

In accordance with one embodiment of the present disclosure, provided is a transgenic mouse cell comprising a nucleic acid encoding a chimeric c-KIT protein, wherein the chimeric c-KIT protein comprises a mouse c-KIT signal peptide, a human c-KIT extracellular domain, a mouse c-KIT transmembrane domain, and a mouse c-KIT intracellular domain.

In some embodiments, the human c-KIT extracellular domain comprises a GNNK sequence. In some embodiments, the human c-KIT extracellular domain comprises the amino acid sequence of SEQ ID NO: 7, 8, 9 or 10, or an amino acid sequence having at least 85% sequence identity to any one of SEQ ID NO: 7, 8, 9 or 10. In some embodiments, the human c-KIT extracellular domain comprises the amino acid sequence of SEQ ID NO: 7, 8, 9 or 10.

In some embodiments, the nucleic acid comprises exogenous and endogenous portions. In some embodiments, the exogenous portion of the nucleic acid comprises coding sequences for the human c-KIT extracellular domain, the mouse c-KIT transmembrane domain, and the mouse c-KIT intracellular domain. In some embodiments, the exogenous portion is inserted to the mouse genome at a locus within exon 2 of the mouse c-KIT gene. In some embodiments, the locus is 3′ to the last codon for the endogenous mouse c-KIT signal peptide. In some embodiments, the exogenous portion further comprises a stop codon. In some embodiments, the insertion is homozygous. In some embodiments, the cell is in vivo in a transgenic mouse.

Further provided, in another embodiments, is a method of testing the activity of an inhibitor to the human c-KIT, comprising contacting a candidate inhibitor with the transgenic mouse cell of the disclosure and measuring c-KIT signaling in the cell.

Also provided is a transgenic mouse comprising the transgenic mouse cell of the present disclosure. Also provided is a method of testing the activity of an inhibitor to the human c-KIT, comprising administering a candidate inhibitor to a transgenic mouse and measuring c-KIT signaling in the mouse.

In some embodiments, the candidate inhibitor is an anti-c-KIT antibody. In some embodiments, the c-KIT signaling is measured with cell proliferation.

Another embodiment provides a method for preparing a transgenic mouse cell, comprising introducing to a target mouse cell a construct comprising (i) a coding sequence encoding a partial chimeric protein comprising a human c-KIT extracellular domain, a mouse c-KIT transmembrane domain, and a mouse c-KIT intracellular domain, and (ii) flanking sequences to enable recombinant integration of the coding sequence to a locus within an exon of an endogenous c-KIT gene in the mouse genome. In some embodiments, the exon is exon 2.

In some embodiments, the transgenic cell expresses a chimeric protein comprising a mouse c-KIT signal peptide, the human c-KIT extracellular domain, the mouse c-KIT transmembrane domain, and the mouse c-KIT intracellular domain.

### BRIEF DESCRIPTION OF THE DRAWINGS

FIG. 1 illustrates the structure of expression vectors tested for functional validation.

FIG. 2 shows the stem cell factor (SCF)-mediated survival and proliferation with mouse or human SCF.

FIG. 3 presents the schematic representation of the c-Kit expression vectors for splicing validation.

FIG. 4A-B illustrate the protocol for in-frame insertion with mouse signal peptide of humanized c-Kit cDNA (hEC-mTM-mIC) (A), and after removal of the neo sequence (B).

FIG. 5A-C present observations in the transgenic mice concerning T cell progenitors in thymus (A), B cell progenitors in the bone marrow (B), and c-Kit expression on bone marrow HSPCs (C).

FIG. 6 shows that briquilimab prevented IgE-Induced PSA in hmCD117 mice. hmCD117 mice were treated with briquilimab, 5 mg/kg, 3 time per week for three weeks or a bolus 25 mg/kg two weeks prior to be sensitized with anti-DNP IgE and challenged 24 hours later with DNP-HSA. Data represent the mean±SEM. Statistical analysis was done by comparison of group means from 0 to 60 min. **p<0.01 by Welch's t-test.

### DETAILED DESCRIPTION

### Definitions

It is to be noted that the term “a” or “an” entity refers to one or more of that entity; for example, “an antibody,” is understood to represent one or more antibodies. As such, the terms “a” (or “an”), “one or more,” and “at least one” can be used interchangeably herein.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis.

The term “isolated” as used herein with respect to cells, nucleic acids, such as DNA or RNA, refers to molecules separated from other DNAs or RNAs, respectively, that are present in the natural source of the macromolecule. The term “isolated” as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to cells or polypeptides which are isolated from other cellular proteins or tissues. Isolated polypeptides is meant to encompass both purified and recombinant polypeptides.

As used herein, an “antibody” or “antigen-binding polypeptide” refers to a polypeptide or a polypeptide complex that specifically recognizes and binds to an antigen. An antibody can be a whole antibody and any antigen binding fragment or a single chain thereof. Thus the term “antibody” includes any protein or peptide containing molecule that comprises at least a portion of an immunoglobulin molecule having biological activity of binding to the antigen. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein.

The terms “antibody fragment” or “antigen-binding fragment”, as used herein, is a portion of an antibody such as F(ab′)₂, F(ab)₂, Fab′, Fab, Fv, scFv and the like. Regardless of structure, an antibody fragment binds with the same antigen that is recognized by the intact antibody. The term “antibody fragment” includes aptamers, spiegelmers, and diabodies. The term “antibody fragment” also includes any synthetic or genetically engineered protein that acts like an antibody by binding to a specific antigen to form a complex.

By “specifically binds” or “has specificity to,” it is generally meant that an antibody binds to an epitope via its antigen-binding domain, and that the binding entails some complementarity between the antigen-binding domain and the epitope. According to this definition, an antibody is said to “specifically bind” to an epitope when it binds to that epitope, via its antigen-binding domain more readily than it would bind to a random, unrelated epitope. The term “specificity” is used herein to qualify the relative affinity by which a certain antibody binds to a certain epitope. For example, antibody “A” may be deemed to have a higher specificity for a given epitope than antibody “B,” or antibody “A” may be said to bind to epitope “C” with a higher specificity than it has for related epitope “D.”

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

As used herein, the term “exogenous” refers to any material introduced from or produced outside an organism, cell, tissue or system.

The term “expand” as used herein refers to increasing in number, as in an increase in the number of T cells. In one embodiment, the T cells that are expanded ex vivo increase in number relative to the number originally present in the culture. In another embodiment, the T cells that are expanded ex vivo increase in number relative to other cell types in the culture. The term “ex vivo,” as used herein, refers to cells that have been removed from a living organism, (e.g., a human) and propagated outside the organism (e.g., in a culture dish, test tube, or bioreactor).

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., sendai viruses, lentiviruses, retroviruses, adenoviruses, and adeno-associated viruses) that incorporate the recombinant polynucleotide.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “transgene” refers to the genetic material that has been or is about to be artificially inserted into the genome of an animal, particularly a mammal and more particularly a mammalian cell of a living animal.

The term “transgenic animal” refers to a non-human animal, usually a mammal, having a non-endogenous (i.e., heterologous) nucleic acid sequence present as an extrachromosomal element in a portion of its cells or stably integrated into its germ line DNA (i.e., in the genomic sequence of most or all of its cells), for example a transgenic mouse. A heterologous nucleic acid is introduced into the germ line of such transgenic animals by genetic manipulation of, for example, embryos or embryonic stem cells of the host animal.

The term “humanized mouse” refers to a mouse that transgenically expresses one or more human genes, or chimeric genes that express at least a portion of a human protein.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Transgenic Mice and Mouse Cells

Inhibitors to the human c-KIT have therapeutic values. For instance, anti-c-KIT antibodies can be used to deplete hematopoietic stem cells (HSC) from the bone marrow of a subject. Deletion of diseased HSC is useful in treating diseases or disorders such as myelodysplastic syndrome (MDS) and acute myeloid leukemia (AML), thereby increasing the relative percentage of healthy HSC.

Anti-c-KIT antibodies are known in the art and/or commercially available, including without limitation, JSP191 (Jasper Therapeutics; Redwood City, CA); CDX-0158 (formerly KTN0158) or CDX-0159 (Celldex Therapeutics, Hampton, NJ); MGTA-117 (AB85) (Magenta Therapeutics, Cambridge, MA); CK6 (Magenta Therapeutics, Cambridge, MA); AB249 (Magenta Therapeutics, Cambridge, MA); and FSI-174 (Gilead, Foster City, CA).

In certain embodiments, the anti-c-KIT antibody binds to the extracellular region of c-KIT, i.e., amino acids 26-524 of SEQ ID NO: 1. The human c-KIT has 8 isoforms, isoforms 1 (NP_000213; SEQ ID NO: 1), isoform 2 (NP_001087241), isoform 3 (NP_001372213; SEQ ID NO: 2), isoform 4 (NP_001372214; SEQ ID NO: 3), isoform 5 (NP_001372215), isoform 6 (NP_001372217), isoform 7 (NP_001372219; SEQ ID NO: 4), and isoform 8 (NP_001372221).

These isoforms are highly homologous. However, four of them (isoforms 1, 3, 4 and 7, Table 1) include a -GNNK- sequence (e.g., amino acid residues 510-514 of SEQ ID NO: 1), while the others don't. Isoforms 1, 3, 4 and 7 are referred to “GNNK” isoforms.

TABLE 1
Sequences

| Name | Sequence |
|---|---|
| Human c-KIT, | MRGARGAWDFLCVLLLLLRVQTGSSQPSVSPGEPSPPSIHPGKSDLIVRVGDEIRLLCTD |
| isoform 1 (SEQ | PGFVKWTFEILDETNENKQNEWITEKAEATNTGKYTCTNKHGLSNSIYVFVRDPAKLFLV |
| ID NO: 1; | DRSLYGKEDNDTLVRCPLTDPEVTNYSLKGCQGKPLPKDLRFIPDPKAGIMIKSVKRAYH |
| NP_000213; | RLCLHCSVDQEGKSVLSEKFILKVRPAFKAVPVVSVSKASYLLREGEEFTVTCTIKDVSS |
| extracellular | SVYSTWKRENSQTKLQEKYNSWHHGDFNYERQATLTISSARVNDSGVFMCYANNTFGSAN |
| portion over | VTTTLEVVDKGFINIFPMINTTVFVNDGENVDLIVEYEAFPKPEHQQWIYMNRTFTDKWE |
| solid lines, | DYPKSENESNIRYVSELHLTRLKGTEGGTYTFLVSNSDVNAAIAFNVYVNTKPEILTYDR |
| transmembrane | LVNGMLQCVAAGFPEPTIDWYFCPGTEQRCSASVLPVDVQTLNSSGPPFGKLVVQSSIDS |
| domain above dotted lines) | 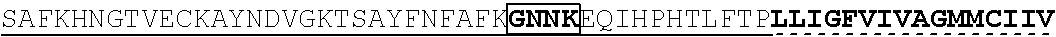 |
| | 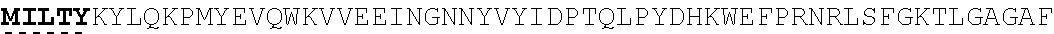 |
| | GKVVEATAYGLIKSDAAMTVAVKMLKPSAHLTEREALMSELKVLSYLGNHMNIVNLLGAC |
| | TIGGPTLVITEYCCYGDLLNFLRRKRDSFICSKQEDHAEAALYKNLLHSKESSCSDSTNE |
| | YMDMKPGVSYVVPTKADKRRSVRIGSYIERDVTPAIMEDDELALDLEDLLSFSYQVAKGM |
| | AFLASKNCIHRDLAARNILLTHGRITKICDFGLARDIKNDSNYVVKGNARLPVKWMAPES |
| | IFNCVYTFESDVWSYGIFLWELFSLGSSPYPGMPVDSKFYKMIKEGFRMLSPEHAPAEMY |
| | DIMKTCWDADPLKRPTFKQIVQLIEKQISESTNHIYSNLANCSPNRQKPVVDHSVRINSV |
| | GSTASSSQPLLVHDDV |
| Human c-KIT, | MRGARGAWDFLCVLLLLLRVQTGSSQPSVSPGEPSPPSIHPGKSDLIVRVGDEIRLLCTD |
| isoform 3 (SEQ | PGFVKWTFEILDETNENKQNEWITEKAEATNTGKYTCTNKHGLSNSIYVFVRDPAKLFLV |
| ID NO: 2; | DRSLYGKEDNDTLVRCPLTDPEVTNYSLKGCQGKPLPKDLRFIPDPKAGIMIKSVKRAYH |
| NP_001372213; | RLCLHCSVDQEGKSVLSEKFILKVRPAFKAVPVVSVSKASYLLREGEEFTVTCTIKDVSS |
| extracellular | SVYSTWKRENSQQTKLQEKYNSWHHGDFNYERQATLTISSARVNDSGVFMCYANNTFGSA |
| portion over | NVTTTLEVVDKGFINIFPMINTTVFVNDGENVDLIVEYEAFPKPEHQQWIYMNRTFTDKW |
| solid lines, | EDYPKSENESNIRYVSELHLTRLKGTEGGTYTFLVSNSDVNAAIAFNVYVNTKPEILTYD |
| transmembrane | RLVNGMLQCVAAGFPEPTIDWYFCPGTEQRCSASVLPVDVQTLNSSGPPFGKLVVQSSID |
| domain above dotted lines) | 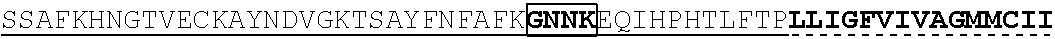 |
| | 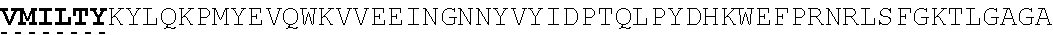 |
| | FGKVVEATAYGLIKSDAAMTVAVKMLKPSAHLTEREALMSELKVLSYLGNHMNIVNLLGA |
| | CTIGGPTLVITEYCCYGDLLNFLRRKRDSFICSKQEDHAEAALYKNLLHSKESSCSDSTN |
| | EYMDMKPGVSYVVPTKADKRRSVRIGSYIERDVTPAIMEDDELALDLEDLLSFSYQVAKG |
| | MAFLASKNCIHRDLAARNILLTHGRITKICDFGLARDIKNDSNYVVKGNARLPVKWMAPE |
| | SIFNCVYTFESDVWSYGIFLWELFSLGSSPYPGMPVDSKFYKMIKEGFRMLSPEHAPAEM |
| | YDIMKTCWDADPLKRPTFKQIVQLIEKQISESTNHIYSNLANCSPNRQKPVVDHSVRINS |
| | VGSTASSSQPLLVHDDV |
| Human c-KIT, | MRGARGAWDFLCVLLLLLRVQTGSSQPSVSPGEPSPPSIHPGKSDLIVRVGDEIRLLCTD |
| isoform 4 (SEQ | PGFVKWTFEILDETNENKQNEWITEKAEATNTGKYTCTNKHGLSNSIYVFVRDPAKLFLV |
| ID NO: 3; | DRSLYGKEDNDTLVRCPLTDPEVTNYSLKGCQGKPLPKDLRFIPDPKAGIMIKSVKRAYH |
| NP_001372214; | RLCLHCSVDQEGKSVLSEKFILKVRPAFKAVPVVSVSKASYLLREGEEFTVTCTIKDVSS |
| extracellular | SVYSTWKRENSQTKLQEKYNSWHHGDFNYERQATLTISSARVNDSGVFMCYANNTFGSAN |
| portion over | VTTTLEVVDKGFINIFPMINTTVFVNDGENVDLIVEYEAFPKPEHQQWIYMNRTFTDKWE |
| solid lines, | DYPKSENESNIRYVSELHLTRLKGTEGGTYTFLVSNSDVNAAIAFNVYVNTKPEILTYDR |
| transmembrane | LVNGMLQCVAAGFPEPTIDWYFCPGTEQRCSASVLPVDVQTLNSSGPPFGKLVVQSSIDS |
| domain above dotted lines) | 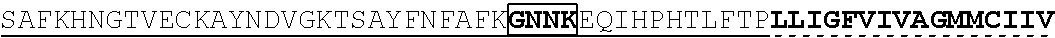 |
| | 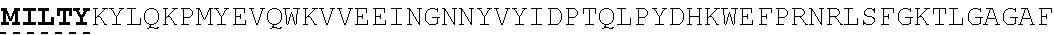 |
| | GKVVEATAYGLIKSDAAMTVAVKMLKPSAHLTEREALMSELKVLSYLGNHMNIVNLLGAC |
| | TIGGPTLVITEYCCYGDLLNFLRRKRDSFICSKQEDHAEAALYKNLLHSKESSCDSTNEY |
| | MDMKPGVSYVVPTKADKRRSVRIGSYIERDVTPAIMEDDELALDLEDLLSFSYQVAKGMA |
| | FLASKNCIHRDLAARNILLTHGRITKICDFGLARDIKNDSNYVVKGNARLPVKWMAPESI |
| | FNCVYTFESDVWSYGIFLWELFSLGSSPYPGMPVDSKFYKMIKEGFRMLSPEHAPAEMYD |
| | IMKTCWDADPLKRPTFKQIVQLIEKQISESTNHIYSNLANCSPNRQKPVVDHSVRINSVG |
| | STASSSQPLLVHDDV |
| Human c-KIT, | MRGARGAWDFLCVLLLLLRVQTGSSQPSVSPGEPSPPSIHPGKSDLIVRVGDEIRLLCTD |
| isoform 7 (SEQ | PGFVKWTFEILDETNENKQNEWITEKAEATNTGKYTCTNKHGLSNSIYVFVRDPAKLFLV |
| ID NO: 4; | DRSLYGKEDNDTLVRCPLTDPEVTNYSLKGCQGKPLPKDLRFIPDPKAGIMIKSVKRAYH |
| NP_001372219; | RLCLHCSVDQEGKSVLSEKFILKVRPAFKAVPVVSVSKASYLLREGEEFTVTCTIKDVSS |
| extracellular | SVYSTWKRENSQQTKLQEKYNSWHHGDFNYERQATLTISSARVNDSGVFMCYANNTFGSA |
| portion over | NVTTTLEVVDKGFINIFPMINTTVFVNDGENVDLIVEYEAFPKPEHQQWIYMNRTFTDKW |
| solid lines, | EDYPKSENESNIRYVSELHLTRLKGTEGGTYTFLVSNSDVNAAIAFNVYVNTKPEILTYD |
| transmembrane | RLVNGMLQCVAAGFPEPTIDWYFCPGTEQRCSASVLPVDVQTLNSSGPPFGKLVVQSSID |
| domain above dotted lines) | 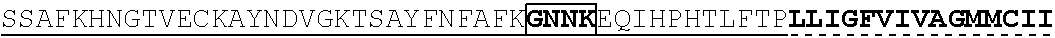 |
| | 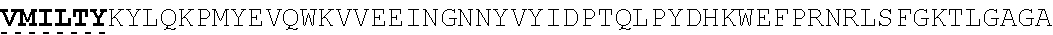 |
| | FGKVVEATAYGLIKSDAAMTVAVKMLKPSAHLTEREALMSELKVLSYLGNHMNIVNLLGA |
| | CTIGGPTLVITEYCCYGDLLNFLRRKRDSFICSKQEDHAEAALYKNLLHSKESSCDSTNE |
| | YMDMKPGVSYVVPTKADKRRSVRIGSYIERDVTPAIMEDDELALDLEDLLSFSYQVAKGM |
| | AFLASKNCIHRDLAARNILLTHGRITKICDFGLARDIKNDSNYVVKGNARLPVKWMAPES |
| | IFNCVYTFESDVWSYGIFLWELFSLGSSPYPGMPVDSKFYKMIKEGFRMLSPEHAPAEMY |
| | DIMKTCWDADPLKRPTFKQIVQLIEKQISESTNHIYSNLANCSPNRQKPVVDHSVRINSV |
| | GSTASSSQPLLVHDDV |
| Mouse c-KIT | MRGARGAWDLLCVLLVLLRGQTATS |
| signal peptide | |
| (SEQ ID NO: 5) | |
| Mouse c-KIT, transmembrane | 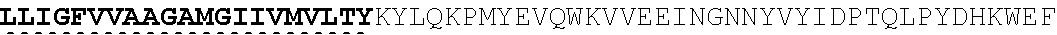 |
| domain and | PRNRLSFGKTLGAGAFGKVVEATAYGLIKSDAAMTVAVKMLKPSAHLTEREALMSELKVL |
| intracellular | SYLGNHMNIVNLLGACTVGGPTLVITEYCCYGDLLNFLRRKRDSFIFSKQEEQAEAALYK |
| domain (SEQ ID | NLLHSTEPSCDSSNEYMDMKPGVSYVVPTKTDKRRSARIDSYIERDVTPAIMEDDELALD |
| NO: 6; | LDDLLSFSYQVAKGMAFLASKNCIHRDLAARNILLTHGRITKICDFGLARDIRNDSNYVV |
| transmembrane | KGNARLPVKWMAPESIFSCVYTFESDVWSYGIFLWELFSLGSSPYPGMPVDSKFYKMIKE |
| domain above | GFRMVSPEHAPAEMYDVMKTCWDADPLKRPTFKQVVQLIEKQISDSTKHIYSNLANCNPN |
| dotted lines) | PENPVVVDHSVRVNSVGSSASSTQPLLVHEDA |
| Human c-KIT | QPSVSPGEPSPPSIHPGKSDLIVRVGDEIRLLCTDPGFVKWTFEILDETNENKQNEWITE |
| isoform 1 | KAEATNTGKYTCTNKHGLSNSIYVFVRDPAKLFLVDRSLYGKEDNDTLVRCPLTDPEVTN |
| extracellular | YSLKGCQGKPLPKDLRFIPDPKAGIMIKSVKRAYHRLCLHCSVDQEGKSVLSEKFILKVR |
| domain (SEQ ID | PAFKAVPVVSVSKASYLLREGEEFTVTCTIKDVSSSVYSTWKRENSQTKLQEKYNSWHHG |
| NO: 7) | DFNYERQATLTISSARVNDSGVFMCYANNTFGSANVTTTLEVVDKGFINIFPMINTTVFV |
| | NDGENVDLIVEYEAFPKPEHQQWIYMNRTFTDKWEDYPKSENESNIRYVSELHLTRLKGT |
| | EGGTYTFLVSNSDVNAAIAFNVYVNTKPEILTYDRLVNGMLQCVAAGFPEPTIDWYFCPG |
| | TEQRCSASVLPVDVQTLNSSGPPFGKLVVQSSIDSSAFKHNGTVECKAYNDVGKTSAYFN |
| | FAFKGNNKEQIHPHTLFTP |
| Human c-KIT | QPSVSPGEPSPPSIHPGKSDLIVRVGDEIRLLCTDPGFVKWTFEILDETNENKQNEWITE |
| isoform 3 | KAEATNTGKYTCTNKHGLSNSIYVFVRDPAKLFLVDRSLYGKEDNDTLVRCPLTDPEVTN |
| extracellular | YSLKGCQGKPLPKDLRFIPDPKAGIMIKSVKRAYHRLCLHCSVDQEGKSVLSEKFILKVR |
| domain (SEQ ID | PAFKAVPVVSVSKASYLLREGEEFTVTCTIKDVSSSVYSTWKRENSQQTKLQEKYNSWHH |
| NO: 8) | GDFNYERQATLTISSARVNDSGVFMCYANNTFGSANVTTTLEVVDKGFINIFPMINTTVF |
| | VNDGENVDLIVEYEAFPKPEHQQWIYMNRTFTDKWEDYPKSENESNIRYVSELHLTRLKG |
| | TEGGTYTFLVSNSDVNAAIAFNVYVNTKPEILTYDRLVNGMLQCVAAGFPEPTIDWYFCP |
| | GTEQRCSASVLPVDVQTLNSSGPPFGKLVVQSSIDSSAFKHNGTVECKAYNDVGKTSAYF |
| | NFAFKGNNKEQIHPHTLFTP |
| Human c-KIT | QPSVSPGEPSPPSIHPGKSDLIVRVGDEIRLLCTDPGFVKWTFEILDETNENKQNEWITE |
| isoform 4 | KAEATNTGKYTCTNKHGLSNSIYVFVRDPAKLFLVDRSLYGKEDNDTLVRCPLTDPEVTN |
| extracellular | YSLKGCQGKPLPKDLRFIPDPKAGIMIKSVKRAYHRLCLHCSVDQEGKSVLSEKFILKVR |
| domain (SEQ ID | PAFKAVPVVSVSKASYLLREGEEFTVTCTIKDVSSSVYSTWKRENSQTKLQEKYNSWHHG |
| NO: 9) | DFNYERQATLTISSARVNDSGVFMCYANNTFGSANVTTTLEVVDKGFINIFPMINTTVFV |
| | NDGENVDLIVEYEAFPKPEHQQWIYMNRTFTDKWEDYPKSENESNIRYVSELHLTRLKGT |
| | EGGTYTFLVSNSDVNAAIAFNVYVNTKPEILTYDRLVNGMLQCVAAGFPEPTIDWYFCPG |
| | TEQRCSASVLPVDVQTLNSSGPPFGKLVVQSSIDSSAFKHNGTVECKAYNDVGKTSAYFN |
| | FAFKGNNKEQIHPHTLFTP |
| Human c-KIT | QPSVSPGEPSPPSIHPGKSDLIVRVGDEIRLLCTDPGFVKWTFEILDETNENKQNEWITE |
| isoform 7 | KAEATNTGKYTCTNKHGLSNSIYVFVRDPAKLFLVDRSLYGKEDNDTLVRCPLTDPEVTN |
| extracellular | YSLKGCQGKPLPKDLRFIPDPKAGIMIKSVKRAYHRLCLHCSVDQEGKSVLSEKFILKVR |
| domain (SEQ ID | PAFKAVPVVSVSKASYLLREGEEFTVTCTIKDVSSSVYSTWKRENSQQTKLQEKYNSWHH |
| NO: 10) | GDFNYERQATLTISSARVNDSGVFMCYANNTFGSANVTTTLEVVDKGFINIFPMINTTVF |
| | VNDGENVDLIVEYEAFPKPEHQQWIYMNRTFTDKWEDYPKSENESNIRYVSELHLTRLKG |
| | TEGGTYTFLVSNSDVNAAIAFNVYVNTKPEILTYDRLVNGMLQCVAAGFPEPTIDWYFCP |
| | GTEQRCSASVLPVDVQTLNSSGPPFGKLVVQSSIDSSAFKHNGTVECKAYNDVGKTSAYF |
| | NFAFKGNNKEQIHPHTLFTP |
| Chimeric c-KIT | MRGARGAWDLLCVLLVLLRGQTATSQPSVSPGEPSPPSIHPGKSDLIVRVGDEIRLLCTD |
| (SEQ ID NO: 11) | PGFVKWTFEILDETNENKQNEWITEKAEATNTGKYTCTNKHGLSNSIYVFVRDPAKLFLV |
| | DRSLYGKEDNDTLVRCPLTDPEVTNYSLKGCQGKPLPKDLRFIPDPKAGIMIKSVKRAYH |
| | RLCLHCSVDQEGKSVLSEKFILKVRPAFKAVPVVSVSKASYLLREGEEFTVTCTIKDVSS |
| | SVYSTWKRENSQTKLQEKYNSWHHGDFNYERQATLTISSARVNDSGVFMCYANNTFGSAN |
| | VTTTLEVVDKGFINIFPMINTTVFVNDGENVDLIVEYEAFPKPEHQQWIYMNRTFTDKWE |
| | DYPKSENESNIRYVSELHLTRLKGTEGGTYTFLVSNSDVNAAIAFNVYVNTKPEILTYDR |
| | LVNGMLQCVAAGFPEPTIDWYFCPGTEQRCSASVLPVDVQTLNSSGPPFGKLVVQSSIDS |
| | 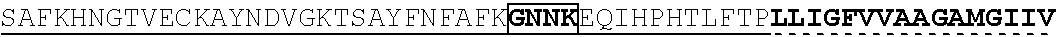 |
| | 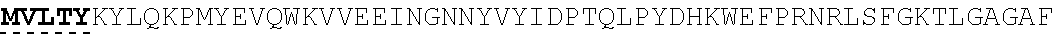 |
| | GKVVEATAYGLIKSDAAMTVAVKMLKPSAHLTEREALMSELKVLSYLGNHMNIVNLLGAC |
| | TVGGPTLVITEYCCYGDLLNFLRRKRDSFIFSKQEEQAEAALYKNLLHSTEPSCDSSNEY |
| | MDMKPGVSYVVPTKTDKRRSARIDSYIERDVTPAIMEDDELALDLDDLLSFSYQVAKGMA |
| | FLASKNCIHRDLAARNILLTHGRITKICDFGLARDIRNDSNYVVKGNARLPVKWMAPESI |
| | FSCVYTFESDVWSYGIFLWELFSLGSSPYPGMPVDSKFYKMIKEGFRMVSPEHAPAEMYD |
| | VMKTCWDADPLKRPTFKQVVQLIEKQISDSTKHIYSNLANCNPNPENPVVVDHSVRVNSV |
| | GSSASSTQPLLVHEDA |
| Exon 2 of mouse | ccacgtctcagccatctgcaagtccaggggagccgtctccgccatccatccatccagcacaatcaga |
| c-KIT (SEQ ID | gttaatagttgaagctggcgacaccctcagcctgacgtgcattgatcccgactttgtcagatggact |
| NO: 12; partial | ttcaagacctatttcaatgaaatggttgagaataaaaaaaatgaatggatccaggaaaaagccgagg |
| signal peptide | ccactcgcacgggcacatacacgtgcagcaacagcaatggcctcacgagttctatttacgtgtttgt |
| underlined) | tagagatcctgccaaacttt |

The instant inventors set out to generate a mouse cell that expresses a chimeric c-KIT protein with a mouse signal peptide (mSP), a human extracellular portion (hEC), a mouse transmembrane domain (mTM), and a mouse intracellular portion (mIC). Initial testing shows that such a chimeric protein was able to bind human SCF (hSCF) and mediate proliferation and survival of the transfected cells (Example 1). This was surprising, even though the chimeric protein indeed was less effective than the human counterpart in mediating SCF signaling, because there was no suggestion that the human extracellular domain would be able to conduct SCF signaling through the mouse transmembrane and intracellular domains.

To make a transgenic cell with the hEC-coding sequence integrated to the genome, the instant inventors designed a cDNA that included the hEC-mTM-mIC portion with flanking sequences to enable insertion of the cDNA to exon 2 of the mouse c-KIT genomic sequence. The mouse signal peptide coding portion ends within exon 2 (Table 1, SEQ ID NO: 12), and thus such an insertion enables expression of a mSP-hEC-mTM-mIC fusion protein when the starting codon of hEC is inserted right after the last codon of mSP, in frame. In another surprising discovery, such insertion did not interrupt the splicing of exon 2, and the fusion protein was expressed as designed. Consequently, transgenic mouse cells and animals were successfully prepared.

In accordance with one embodiment of the present disclosure, provided is a transgenic mouse cell comprising a nucleic acid encoding a chimeric c-KIT protein, wherein the chimeric c-KIT protein comprises a mouse c-KIT signal peptide, a human c-KIT extracellular domain, a mouse c-KIT transmembrane domain, and a mouse c-KIT intracellular domain. In some embodiments, the chimeric c-KIT protein is able to bind human SCF. In some embodiments, the chimeric c-KIT protein is able to mediate SCF signaling.

In some embodiments, the human c-KIT extracellular domain (hEC) includes the extracellular domain of any of the known human c-KIT isoforms, such as isoforms 1-8, or their biological equivalent. A biological equivalent of a reference sequence (e.g., SEQ ID NO: 7) includes those having at least 70%, 75%, 80%, 85%, 90%, 95%, 96%, 97%, 98%, or 99% sequence identity to the reference sequence.

Some of the human c-KIT isoforms include a -GNNK- sequence (e.g., amino acid residues 510-514 of SEQ ID NO: 1) in the extracellular domain, while the others don't. In a preferred embodiment, the hEC includes the extracellular domain of one of the “GNNK” isoforms (isoforms 1, 3, 4 or 7). The full protein sequences of isoforms 1, 3, 4 and 7 are provided in Table 1 as SEQ ID NO: 1-4, and their extracellular domains are provided as SEQ ID NO: 7-10.

In one embodiment, the c-KIT extracellular domain (hEC) includes the amino acid sequence of SEQ ID NO: 7 or an amino acid sequence having at least 70%, 75%, 80%, 85%, 90%, 95%, 96%, 97%, 98%, or 99% sequence identity to SEQ ID NO: 7. In one embodiment, the c-KIT extracellular domain (hEC) includes the amino acid sequence of SEQ ID NO: 8 or an amino acid sequence having at least 70%, 75%, 80%, 85%, 90%, 95%, 96%, 97%, 98%, or 99% sequence identity to SEQ ID NO: 8. In one embodiment, the c-KIT extracellular domain (hEC) includes the amino acid sequence of SEQ ID NO: 9 or an amino acid sequence having at least 70%, 75%, 80%, 85%, 90%, 95%, 96%, 97%, 98%, or 99% sequence identity to SEQ ID NO: 9. In one embodiment, the c-KIT extracellular domain (hEC) includes the amino acid sequence of SEQ ID NO: 10 or an amino acid sequence having at least 70%, 75%, 80%, 85%, 90%, 95%, 96%, 97%, 98%, or 99% sequence identity to SEQ ID NO: 10. An example amino acid sequence of the chimeric protein is provided in SEQ ID NO: 11 (Table 1), which includes the extracellular domain of human isoform 1.

In some embodiments, the c-KIT transmembrane domain (mTM) and intracellular domain (mIC) includes the amino acid sequence of SEQ ID NO: 6 or an amino acid sequence having at least 70%, 75%, 80%, 85%, 90%, 95%, 96%, 97%, 98%, or 99% sequence identity to SEQ ID NO: 6.

The chimeric c-KIT, in some embodiments, is encoded by a nucleic acid integrated to the genome of the transgenic cell. The nucleic acid can include an exogenous portion and an endogenous portion. As tested, a cDNA that encodes the hEC-mTM-mIC portion of the chimeric c-KIT protein can be inserted to one of the exons of the endogenous mouse c-KIT gene.

In one embodiment, the insertion is at a locus within exon 2 of the mouse c-KIT gene. In a preferred embodiment, the cDNA encodes hEC-mTM-mIC and the first codon of the hEC is inserted immediately after the last codon (nucleotides 6-8 of SEQ ID NO: 12, which shows the exon 2 genomic sequence) of the mouse signal peptide (SEQ ID NO: 5).

In an alternative embodiment, the insertion is at a locus within exon 2 but more downstream. Such would allow inclusion of some amino acid(s) of the mouse extracellular domain in the expressed chimeric protein. However, it is contemplated that the addition of a small number of mouse amino acids would not impact the activity of the chimeric protein. Also, given the homology between the mouse and human c-KIT protein, when the additional mouse extracellular amino acids are included, some of the human ones (N-terminal) can be optionally deleted from the cDNA to neutralize the impact.

In yet another alternative embodiment, the insertion is at a locus within exon 2 but more upstream from the end of the last codon of the signal peptide. To compensate for the partial truncation of the C-terminus of the mouse signal peptide, the cDNA can further include the truncated portion, rendering the expressed chimeric protein whole.

Likewise, in another embodiment, the insertion is at a locus within exon 1 which is upstream from the last codon of the signal peptide. To compensate for the partial truncation of the C-terminus of the mouse signal peptide, the cDNA can further include the truncated portion, rendering the expressed chimeric protein whole.

In some embodiments, the cDNA further includes a stop codon. In some embodiments, the cDNA further includes a poly(A) signal. In some embodiments, the cDNA further includes a selection marker, such as coding sequences for a neomycin resistant protein or a fluorescence protein. Such markers can facilitate selection of successfully integrated cells. They can be excised from the genome with a known technology (e.g., Cre-Lox recombination) upon completion of the selection. In some embodiments, the cDNA is codon-optimized. In some embodiments, the insertion is heterozygous. In some embodiments, the insertion is homozygous.

The general methods for constructing recombinant DNA which can be introduced into target cells are well known to those skilled in the art, and the same compositions and methods of construction may be utilized to produce the DNA useful herein. For example, J. Sambrook et al., Molecular Cloning: A Laboratory Manual, Cold Spring Harbor Laboratory Press (2d ed., 1989), provides suitable methods of construction. The recombinant DNA can be readily introduced into the target cells, i.e., totipotent cells such as fertilized eggs, by methods well known to the art.

If the target cell is a stem cell, such as an embryonic stem cell, a transgenic mouse can be generated. Accordingly, the present disclosure also provides transgenic mice having at least a transgenic cell as described herein. In some embodiments, the transgenic cell is heterozygous with respect to the chimeric protein. In some embodiments, the transgenic cell is homozygous.

Methods for using the transgenic cell or mouse of the disclosure are also provided. In one embodiment, provided is a method of testing the activity of an inhibitor to the human c-KIT, which entails contacting a candidate inhibitor with a transgenic mouse cell as described here, and measuring c-KIT signaling in the cell. Also provided is a method of testing the activity of an inhibitor to the human c-KIT, which entails administering a candidate inhibitor to a transgenic mouse, and measuring c-KIT signaling in the mouse. In some embodiments, the candidate inhibitor is an anti-c-KIT antibody.

c-KIT signaling can be measured with methods known in the art. C-KIT is a cytokine receptor which binds to stem cell factor (SCF). The binding activates c-KIT's intrinsic tyrosine kinase activity, that in turn phosphorylates and activates signal transduction molecules that propagate the signal in the cell. Signaling through KIT plays a role in cell survival, proliferation, and differentiation. In some embodiments, the inhibitor activity can be measured with cell proliferation or differentiation, as demonstrated in the accompanying experimental examples.

### EXAMPLES

### Example 1: Generation and Testing of Transgenic Ba/F3 Cells

This example tested the effect of expression of exogenous c-KIT cDNA on the proliferation and survival of Ba/F3 cells.

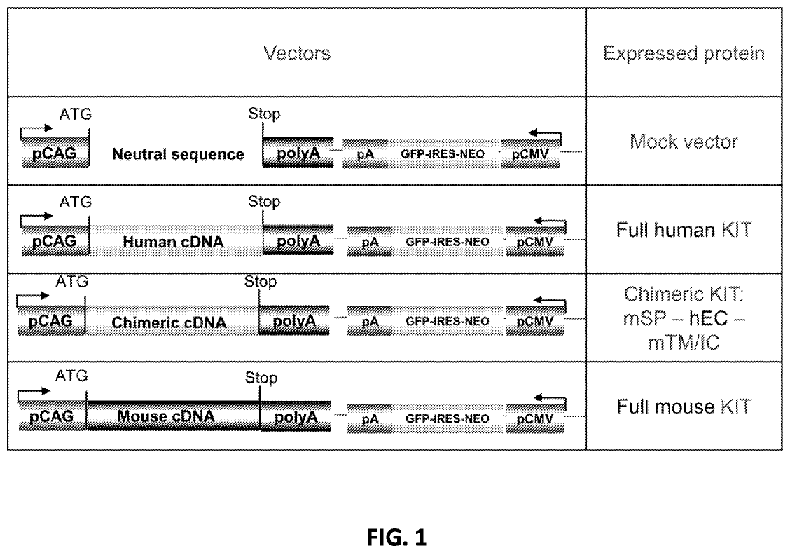

Expression vectors that included (A) a human c-KIT cDNA, (B) a mouse c-KIT cDNA, (C) a chimeric human/mouse c-KIT cDNA (mSP-hEC-mTM-mIC), or (D) a mock control were constructed. The chimeric c-KIT cDNA encodes a chimeric c-KIT protein that included a mouse signal peptide (mSP), a human extracellular portion (hEC), a mouse transmembrane domain (mTM), and a mouse intracellular portion (mIC). Each expression vector further included sequences for expressing GFP and a neomycin resistant gene for cell sorting and selection (FIG. 1). Each vector was transfected into a Ba/F3 cell, and successfully transfected cells were harvested.

Stably transfected Ba/F3 cells were starved overnight and were stimulated with various concentrations of mouse or human SCF (stem cell factor) for 4 days. Cell survival was assessed by DAPI exclusion flow cytometry. Cell proliferation was assessed by EdU flow cytometry.

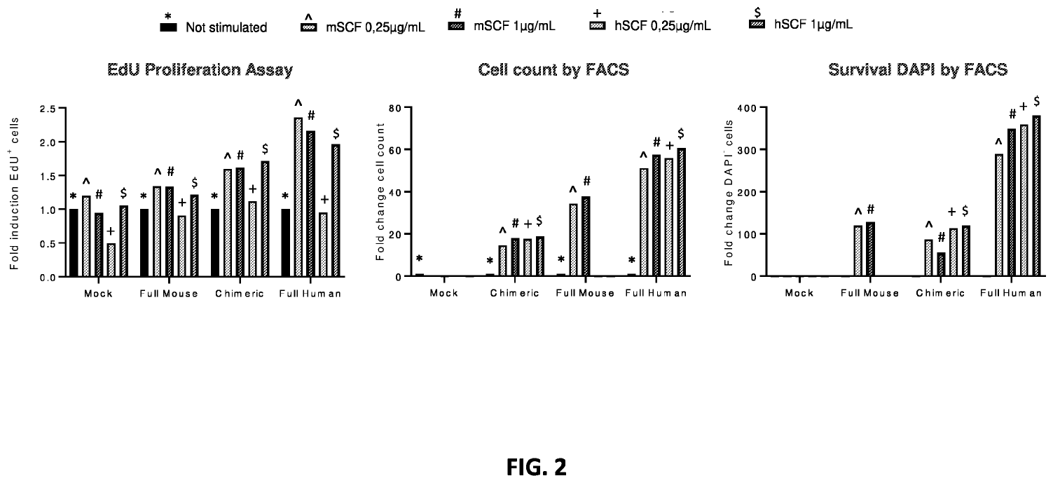

As shown in FIG. 2, mouse SCF (mSCF) stimulation induced proliferation and survival of all vector-transfected mouse cells, demonstrating that both human and chimeric c-KIT proteins were able to mediate mSCF binding and signaling. Meanwhile, human SCF (hSCF) stimulation induced proliferation and survival of only Ba/F3 cells transfected with either the human or chimeric c-KIT expression vectors. It was observed, however, that the chimeric c-KIT protein was less effective, than the human counterpart, in mediating SCF signaling.

### Example 2. In Vitro Splicing Testing

It was proposed that a chimeric c-KIT cDNA is preferably inserted to the endogenous c-KIT locus of the mouse genome in the target cell. It was suspected, however, such insertion would lead to modification of the exonic splicing regulatory sequences, causing disruption to the inserted cDNA. This example tested whether insertion of a chimeric cDNA to the c-KIT locus in the mouse genome would lead to altered splicing.

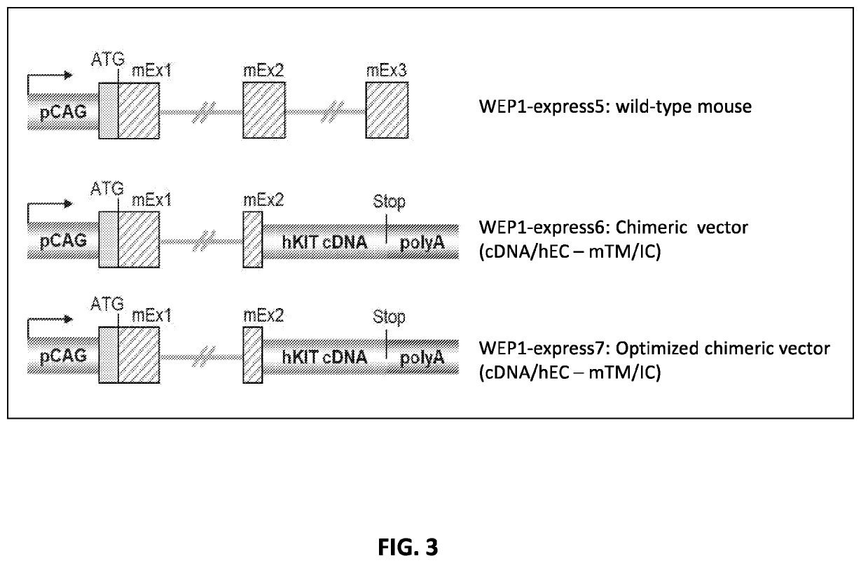

The cDNA encoded a chimeric c-KIT protein (hEC-mTM-mIC) that included the human extracellular portion (hEC) and mouse transmembrane domain (mTM)/intracellular portion (mIC), in addition to a stop codon and poly(A) sequences (FIG. 3). A slightly different version of the cDNA was also prepared, which was codon-optimized. Homologous recombination was designed such that the cDNA was inserted, in frame, into exon 2 of the mouse c-KIT gene and following the mouse signal peptide (mSP). Therefore, if exon 1 and exon 2 were spliced correctly, the transcript would include the desired mSP-hEC-mTM-mIC chimeric protein.

The constructs were transfected to 3T3 murine cells or embryonic stem (ES) cell. 24 hours post-transfection, reverse transcription was performed on extracted RNA, and the cDNA were amplified. Gel electrophoresis with the amplified PCR products confirmed that the correct splicing occurred in the transfected cells.

### Example 3. Generation and Testing of Transgenic Mice

Using a procedure similar to Example 2, this example prepared murine ES cells into which chimeric hEC-mTM-mIC cDNA was stably integrated to the genome. The transgenic ES cell clones can be implanted into blastocyst-stage embryos for generation of chimeras.

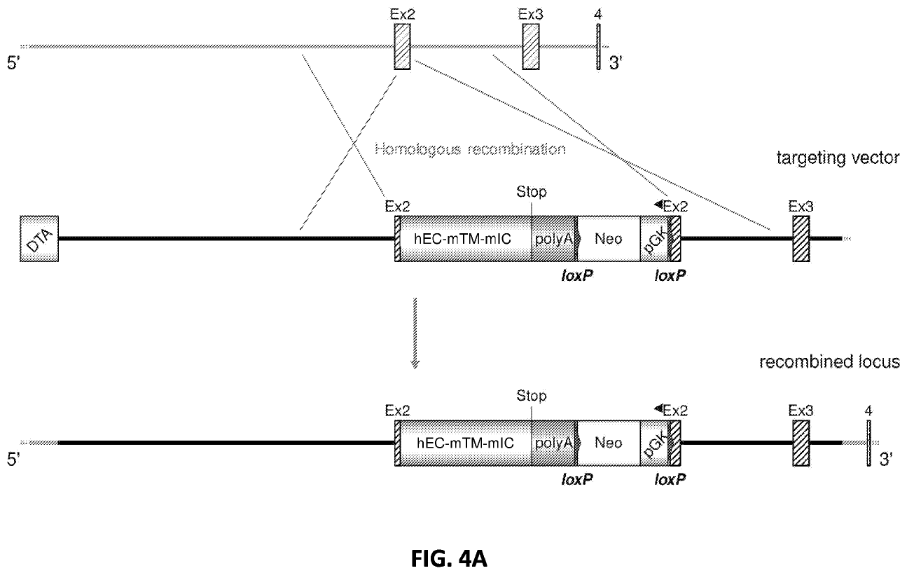

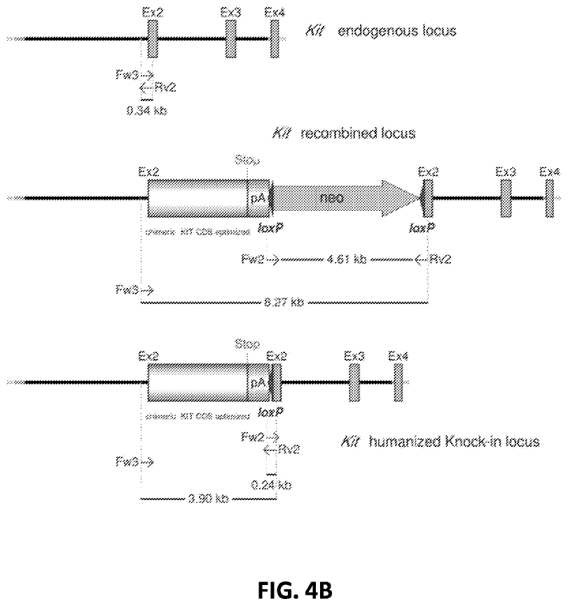

As illustrated in FIG. 4A, the construct included, in addition to hEC-mTM-mIC, stop codon, poly(A), and a neomycin resistant gene (Neo). The hEC sequence was GNNK(+). The neomycin resistant gene was flanked by a pair of loxP sites. The neomycin resistant gene allowed selection of transfected cells, which was then excised with (FIG. 4B). Insertion of the exogenous sequences was confirmed with PCT amplification with suitable primers (as illustrated in FIG. 4B).

To produce highly chimeric male mice carrying the recombined locus, transfected and integrated ES cell clones were in blastocysts. The ES cells were derived from the inner cell mass of 3.5 days old embryos (blastocyst stage). These cells are pluripotent and thus, when implanted into blastocyst-stage embryos, are able to contribute to every cell lineage, including the germ layer. These blastocysts were implanted in pseudo-pregnant females that are allowed to develop to term. Chimerism rate was then assessed in the progeny by coat color markers comparison.

Out of the 115 pups born, 23 (20%) were homozygous (10 males and 13 females) and all harbored white patches on their black fur; 65 (57%) were heterozygous (29 males and 36 females); among them, 34 harbored white patches (15 males and 19 females), 31 were black (14 males and 17 females); and 27 (23%) were wild-types (17 males and 10 females) and all were black. No differences was observed between WT and c-KIT heterozygous mice in terms of cellularity, viability and T/B cells progenitors proportions in the thymus and bone marrow.

Human c-Kit was detected in c-KIT heterozygous progenitors using anti-c-Kit antibodies and at similar level to mouse c-Kit on WT mice. Similar proportions of lineage negative cells, multiple progenitor and LSK were observed in c-KIT heterozygous and WT mice.

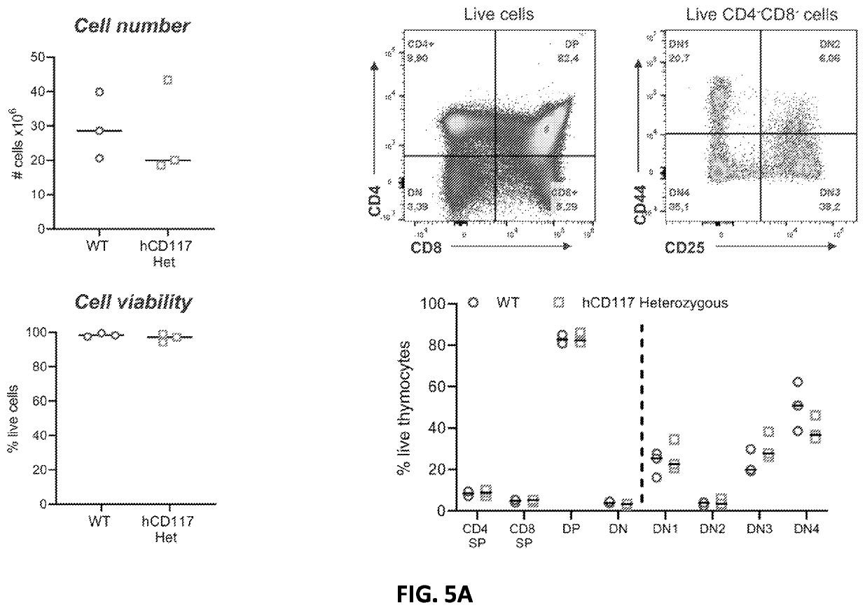

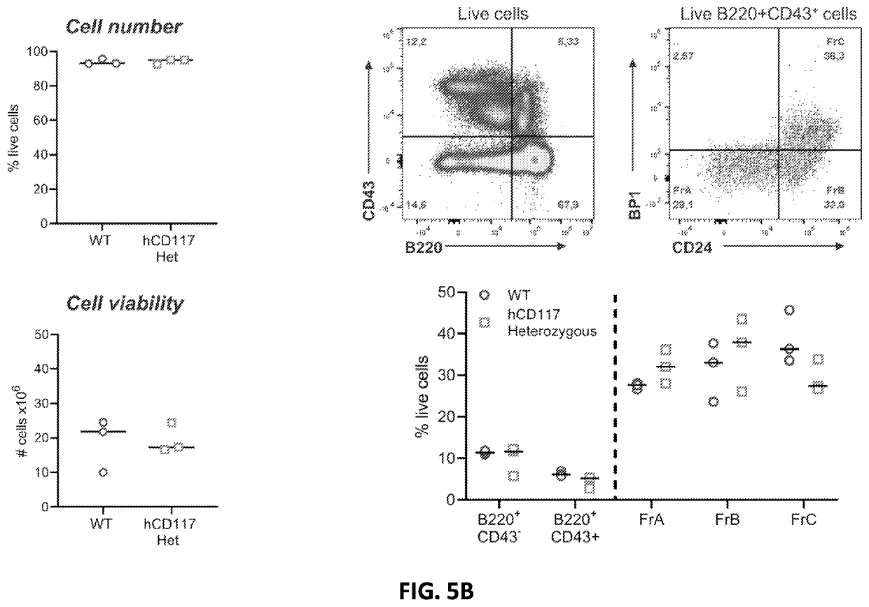

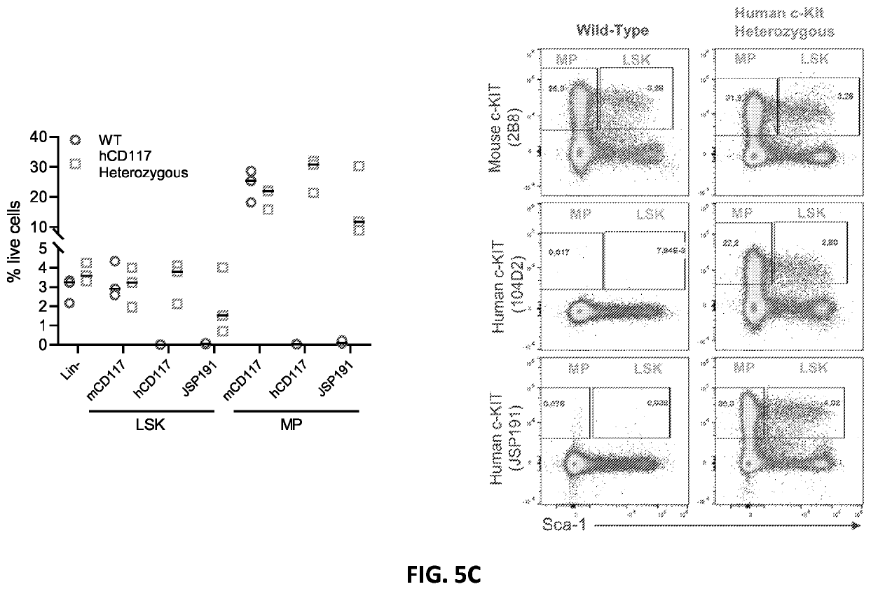

Cell number was slightly lower in the thymus (FIG. 5A) and bone marrow (FIG. 5B) of c-KIT homozygous mice as compared to WT mice. No differences was observed between WT and c-KIT homozygous mice in terms of viability and T/B cells progenitors proportions (FIG. 5C) in the thymus and bone marrow.

### Example 4. Testing of Therapeutics in Transgenic Mice

Stem Cell Factor (SCF) signaling through c-Kit (CD117) plays a key role in mast cell differentiation and survival. Inhibition of this pathway has the potential to treat mast cell-mediated disorders. This example tested, in a transgenic mouse model as prepared in Example 3, whether briquilimab, a humanized aglycosylated monoclonal antibody against CD117, can inhibit SCF signaling and deplete human mast cells.

Methods: The mouse model expressed the chimeric CD117 (hmCD117) as tested in Example 3. Passive systemic anaphylaxis (PSA) was induced by IgE. The pharmacokinetics, pharmacodynamics, and effect on PSA response of briquilimab were evaluated in hmCD117 mice.

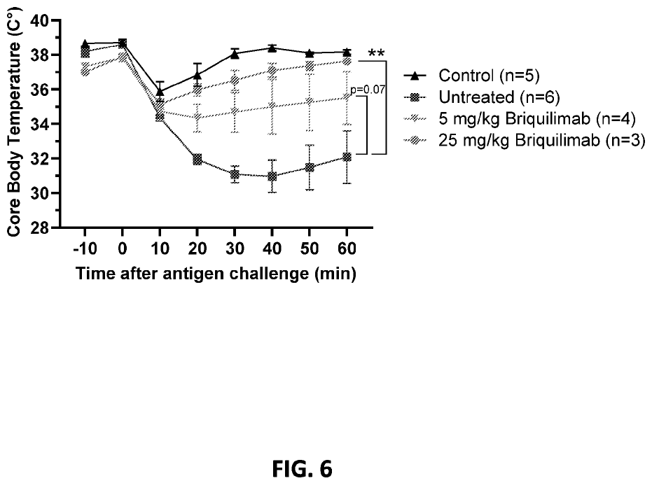

Results: Chimeric hmCD117 was responsive to mouse SCF and was recognized by briquilimab. Treatment with briquilimab (5, 10, and 25 mg/kg, I.V.) in hmCD117 mice caused transient modest decreases in peripheral blood counts and transient depletion of CD117 expressing hematopoietic stem cells. The pharmacokinetic clearance of briquilimab in the serum was dose dependent in a nonlinear manner. Briquilimab treatment with 5 mg/kg, 3 times per week for three weeks, followed by IgE-induced PSA challenge, led to reduced anaphylactic response. Notably, briquilimab treatment with a single 25 mg/kg dose two weeks before PSA challenge completely prevented anaphylactic response, suggesting a single high dose of briquilimab effectively blocks PSA response in hmCD117 mice (FIG. 6).

The present disclosure is not to be limited in scope by the specific embodiments described which are intended as single illustrations of individual aspects of the disclosure, and any compositions or methods which are functionally equivalent are within the scope of this disclosure. It will be apparent to those skilled in the art that various modifications and variations can be made in the methods and compositions of the present disclosure without departing from the spirit or scope of the disclosure. Thus, it is intended that the present disclosure cover the modifications and variations of this disclosure provided they come within the scope of the appended claims and their equivalents.

All publications and patent applications mentioned in this specification are herein incorporated by reference to the same extent as if each individual publication or patent application was specifically and individually indicated to be incorporated by reference.

What is claimed is:

1. A transgenic mouse cell comprising a nucleic acid encoding a chimeric c-KIT protein, wherein the chimeric c-KIT protein comprises a mouse c-KIT signal peptide, a human c-KIT extracellular domain, a mouse c-KIT transmembrane domain, and a mouse c-KIT intracellular domain.

2. The transgenic mouse cell of claim 1, wherein the human c-KIT extracellular domain comprises a GNNK sequence.

3. The transgenic mouse cell of claim 2, wherein the human c-KIT extracellular domain comprises the amino acid sequence of SEQ ID NO: 7, 8, 9 or 10, or an amino acid sequence having at least 85% sequence identity to any one of SEQ ID NO: 7, 8, 9 or 10.

4. The transgenic mouse cell of claim 2, wherein the human c-KIT extracellular domain comprises the amino acid sequence of SEQ ID NO: 7, 8, 9 or 10.

5. The transgenic mouse cell of any preceding claim, wherein the nucleic acid comprises exogenous and endogenous portions.

6. The transgenic mouse cell of claim 5, wherein the exogenous portion of the nucleic acid comprises coding sequences for the human c-KIT extracellular domain, the mouse c-KIT transmembrane domain, and the mouse c-KIT intracellular domain.

7. The transgenic mouse cell of claim 6, wherein the exogenous portion is inserted to the mouse genome at a locus within exon 2 of the mouse c-KIT gene.

8. The transgenic mouse cell of claim 7, wherein the locus is 3′ to the last codon for the endogenous mouse c-KIT signal peptide.

9. The transgenic mouse cell of any one of claims 6-8, wherein the exogenous portion further comprises a stop codon.

10. The transgenic mouse cell of any one of claims 7-9, wherein the insertion is homozygous.

11. The transgenic mouse cell of any preceding claim, wherein the cell is in vivo in a transgenic mouse.

12. A transgenic mouse comprising the transgenic mouse cell of any one of claims 1-11.

13. A method of testing the activity of an inhibitor to the human c-KIT, comprising contacting a candidate inhibitor with the transgenic mouse cell of any one of claims 1-11 and measuring c-KIT signaling in the cell.

14. A method of testing the activity of an inhibitor to the human c-KIT, comprising administering a candidate inhibitor to a transgenic mouse of claim 12, and measuring c-KIT signaling in the mouse.

15. The method of claim 13 or 14, wherein the candidate inhibitor is an anti-c-KIT antibody.

16. The method of any one of claims 13-15, wherein the c-KIT signaling is measured with cell proliferation.

17. A method for preparing a transgenic mouse cell, comprising introducing to a target mouse cell a construct comprising (i) a coding sequence encoding a partial chimeric protein comprising a human c-KIT extracellular domain, a mouse c-KIT transmembrane domain, and a mouse c-KIT intracellular domain, and (ii) flanking sequences to enable recombinant integration of the coding sequence to a locus within an exon of an endogenous c-KIT gene in the mouse genome.

18. The method of claim 17, wherein the exon is exon 2.

19. The method of claim 17 or 18, wherein the transgenic cell expresses a chimeric protein comprising a mouse c-KIT signal peptide, the human c-KIT extracellular domain, the mouse c-KIT transmembrane domain, and the mouse c-KIT intracellular domain.
